(19) United States
(12) Patent Application Publication
(10) Pub. No.: US 2010/0150805 A1
(43) Pub. Date: Jun. 17, 2010

(54) HIGHLY STABLE AND REFRACTORY MATERIALS USED AS CATALYST SUPPORTS

(75) Inventors: Manuela Serban, Glenview, IL (US); Alakananda Bhattacharyya, Glen Ellyn, IL (US); Kurt M. Vanden Bussche, Lake in the Hills, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(73) Assignee: UOP LLC, Morristown, NJ (US)

(21) Appl. No.: 12/555,365

(22) Filed: Sep. 8, 2009

Related U.S. Application Data
(60) Provisional application No. 61/138,156, filed on Dec. 17, 2008.

(51) Int. Cl. B01D 53/48; C07C 5/333; C07C 5/05; C07D 301/03; C07D 301/10; C07C 57/03; C07C 51/36

(52) U.S. Cl. 423/244.02; 585/654; 585/271; 549/523; 549/534; 562/598; 562/592

(57) ABSTRACT

This invention involves highly porous, stable metal oxide felt materials that are used as catalytic supports for a number of different applications including dehydrogenation of light paraffins to olefins, selective hydrogenation of dienes to olefins, hydrogenation of carboxylic acids, oxidation or ammoxidation reactions, epoxidation of light olefins and removal of sulfur compounds from gas streams.

### CROSS-REFERENCE TO RELATED APPLICATION

This application claims priority from Provisional Application No. 61/138,156 filed Dec. 17, 2008, the contents of which are hereby incorporated by reference.

### BACKGROUND OF THE INVENTION

For any heterogeneous catalytic applications, the support used to disperse the active catalytic phase is a critical element for the overall success of the catalytic system. Supports anchor the catalyst materials and the distribution of the catalyst active sites depends on the physical properties of the supports. Overall performance and the life of a catalyst system often depend on the nature and composition of the supports. In particular, for high temperature applications under corrosive environments, the catalytic system has to be thermally stable and resistant to repeated temperature cycling as well as inert to chemical attack by the reaction media.

### SUMMARY OF THE INVENTION

This invention discloses processes for the use of a highly porous, stable and refractory class of materials, namely (metal) oxide felts, such as, ZrO₂, Ce₂O₃, CeO₂, Y₂O₃, TiO₂, HfO₂, Al₂O₃, Nb₂O₅, La₂O₃, Sm₂O₃, Yb₂O₃, and the like and their combinations used as catalytic supports for different varied applications. Two major physical properties of a catalyst support are surface area and pore volume. Although these properties are not changed in the felt materials, the stacking property of the felt materials is changed drastically; from the natural gravity based stacking of traditional catalysts packed in a fixed bed reactor that creates a certain void space, to a flexible, controlled, fabricated stacking for the felt materials. This may allow effective interaction of the feed with the catalyst. Also, the flexibility of the felt openings is significantly different than the rigid porous structure of a traditional catalyst, fact that could offer unique properties to the felt catalyst. For example, if the felt material is used as an absorbent in which the physical dimension of the phases generated during absorption and regeneration are very different, e.g., oxide and sulfide phases, the flexible felt porous structure could allow repeated cycling between smaller and bulkier phases without weakening the support porous structure.

### DETAILED DESCRIPTION OF THE INVENTION

The potential applications for these felt materials used as catalytic supports may be in the areas of low, medium or high temperature catalytic processes, under an oxidizing or reducing environment, under highly acidic or basic conditions, in gas, liquid, or mixed phases. What makes this invention unique is the support used to disperse the active catalytic phase. The support is a highly porous, stable and refractory ceramic textile composed entirely of inorganic fibers. For example, zirconia felts are composed of 100%, 4-6 micron diameter 10% yttria-stabilized zirconia fibers which are mechanically interlocked to give a light weight, very flexible and porous media. The zirconia felts can be used in extremely corrosive environments, they are stable in strong oxidizing or reducing conditions, and are not reactive to alkali vapors or salts. They undergo no phase transition on temperature cycling and are capable of use at temperatures in excess of 1500° C. Similarly, ceria felts have high surface area, high temperature stability and excellent thermal shock resistance. Some properties of the zirconia and ceria felts are given in the following Table. In addition to zirconia and ceria, it is possible to fabricate other refractory (metal) oxide felts, such as Y₂O₃, TiO₂, HfO₂, Al₂O₃, Nb₂O₅, La₂O₃, Yb₂O₃, and mixed oxide felts like Al₂O₃—SiO₂, HfO₂—CeO₂, Sm₂O₃—CeO₂, Yb₂O₃—CeO₂. The metal oxide felt material comprises layers having a thickness from about 0.25 to about 6.35 mm. The metal oxide felt material has a bulk porosity from about 50 to 100% and preferably from about 88 to 96%. The metal oxide felt material has a bulk density of about 128 to 1073 grams/liter and a melting point between about 1500° and 5000° C.

TABLE

| Property | Zirconia Felts | Ceria Felts |
|---|---|---|
| Bulk Porosity (%) | 88-96 | 90-96 |
| Bulk Density (g/cm³) | 0.24-0.48 | 0.24-0.69 |
| Melting Point (° C.) | 4700 | 2590 |
| Minimum wrapping diameter | 0.25-3 | — |
| before breaking (inch) | | |
| % Shrinkage after 1 h at | 1.5-5 | 6-9 |
| 1650° C. | | |

The catalytic active phase supported on the above porous felts for any given catalytic application could be any metals, mixed metals, i.e., Pt, Pd, Rh, Ag, metal oxides or mixed metal oxides of Zn, Fe, Ni, Co, Cu, Ce, Ba, Ca, Mo, Mn, Mg, Ti, V, W and their mixtures dispersed on the above oxide felts using any of the methods known in the art, i.e., wet impregnation and subsequent calcination and/or reduction, metal vapor deposition and subsequent metal oxidation.

This class of highly porous, stable and refractory materials can be used as catalytic supports for any given catalytic application at low, medium or high temperature, under an oxidizing or reducing environment, under highly acidic or basic conditions in gas, liquid, or mixed phases. Without wanting to be exclusive, some examples of such applications and their corresponding catalysts include the following catalytic materials.

Noble metals, i.e., palladium, ruthenium, rhodium, osmium, iridium, and platinum and selected promoters and stabilizers, can be supported on these felt supports for the dehydrogenation of light paraffins, i.e., ethane, propane, butane, to their corresponding olefins. Generally, the concentration of noble metal will range from about 0.01 to about 2 wt-% and the promoter from about 0.1 to 4 wt-%. The reaction temperatures can range from about 400° to 800° C. at pressures less than 2 atmospheres.

Noble metals, i.e., palladium, ruthenium, rhodium, osmium, iridium and platinum and selected promoters such as copper, silver, gold, zinc, cadmium and mercury and stabilizers, supported on these felt supports can be used for the selective hydrogenation of dienes (acetylene or propadiene) to their corresponding olefins (ethylene or propylene). The concentration of noble metal will range from about 0.01 to 2 wt-% and the promoter from about 0.01 to 4 wt-%. The reaction temperatures can range from about 0° to 100° C. at pressures greater than about 2 atmospheres.

Noble metals, i.e., palladium, ruthenium, rhodium, osmium, iridium and platinum and selected promoters and stabilizers, i.e., rhenium, ruthenium, tin, iron, silver, cobalt, manganese, and molybdenum supported on these felt supports can be used for the hydrogenation of monocarboxylic, dicarboxylic, or multicarboxylic acids. For example, these reactions include the hydrogenation of maleic acid to produce tetrahydrofuran and 1,4, buthanediol and mixtures thereof. The Noble metal is present in concentrations of about 0.05 to 5 wt-% and the promoter present from about 1 to 10 wt-%. The reaction temperatures can range from about 50° to 300° C. at pressures from about 20 to 400 atmospheres.

Redox mixed metal oxides such as molybdenum, vanadium, antimony, bismuth and the like on stable and inert felts as described above can be used for the oxidation of hydrocarbons. For example, the oxidation or ammoxidation of butane to produce acrylic acid and acrylonitrile, respectively, may be performed. The total metals ranges from 10 to 60 wt-%. The reaction temperatures can range from about 200° to 600° C. at pressures from about 1 to 4 atmospheres.

Silver and selected promoters like alkali or alkaline earth chloride salts supported on these felts can be used for the epoxidation of light olefins. For example, oxidation of ethylene to ethylene oxide. The metal concentration of silver may range from about 3 to 25 wt-%. The reaction temperatures can range from about 150° to 250° C. at pressures from about 7 to 33 atmospheres.

Metal or metal oxides of manganese, zinc, iron, copper, nickel or any other metal oxides with favorable thermodynamics for the metal sulfide phase formation in a reducing environment supported on these felt supports for the removal of S-compounds from any gas stream containing S-compounds, e.g., removal of H₂S and COS compounds from a reducing fuel gas originating from a coal gasifier. In these reactions, about 10 to 100% of the metal oxide is converted to metal sulfide. Metal or metal oxides of Mg, Ce or any other metal oxides with favorable thermodynamics for the metal sulfate phase formation in an oxidizing environment supported on these felt supports can be used for the removal of S-compounds from any gas stream containing S-compounds, e.g., removal of SOₓ from oxidizing FCC flue gases. In these reactions, about 10 to 100% of metal oxides are converted to metal sulfates. The total metals can range from about 10 to 60 wt-%. The reaction temperatures may range from about 250° to 950° C. at pressures from about 1 to 100 atmospheres. An example of how to make use of this invention is provided below.

A manganese oxide supported on yttria stabilized zirconia felt was prepared via the wet impregnation technique and calcined at 800° C. under air. The Mn loading was 22 wt-%. The Mn oxide supported on yttria stabilized zirconia felt catalyst showed characteristic lines at 23.2±0.5 deg. 2-theta, 28.942±0.5 deg. 2-theta, 30.220±0.5 deg. 2-theta, 33.039±0.5 deg. 2-theta, 35.060±0.5 deg. 2-theta, 38.303±0.5 deg. 2-theta, 45.243±0.5 deg. 2-theta, 49.441±0.5 deg. 2-theta, 50.318±0.5 deg. 2-theta, 55.261±0.5 deg. 2-theta, 57.024±0.5 deg. 2-theta, 59.779±0.5 deg. 2-theta, 62.779±0.5 deg. 2-theta, 65.841±0.5 deg. 2-theta, under X-Ray Diffraction. High resolution Scanning Electron Microscopy (SEM) image reveals that the metal oxide coats the fibers of the zirconia felt uniformly and a Backscattered Electron image of a cross-section of the metal oxide on the felt material mounted on a resin indicates that the metal oxide layer is very porous. This material was used as a sulfur scavenger from a fuel gas simulating an air-blown gasifier containing 1.35% H₂S+13.3% H₂+13.14% CO+13.5% CO₂+59% N₂. The sulfidation step was done at 750° C. and 1600 h⁻¹ space velocity. Under these reducing conditions, the active oxide phase for the sulfidation reaction is Mn(II)O. The regeneration was performed in-situ with lean air (2% O₂ in N₂) at 800° C. and 1600 h⁻¹ space velocity. The Mn-zirconia felt sorbent can easily be cycled between the oxide and sulfide phases with 100% S uptake.

After the six cycles test, the zirconia felt structure remained intact and the only manganese phase detected was MnS with no MnO left behind. The XRD spectra of the five times oxidized material indicates that the sulfided Mn was completely oxidized to Mn₂O₃ (which is further fully reduced to Mn(II)O in the presence of the reducing fuel gas during the sulfidation cycle). The metal oxide supported on this felt material has more sulfur absorbing capacity than metal on traditional bulk zirconia, freshly precipitated or amorphous.

1. A catalytic process comprising contacting a feed with a composite material comprising a support structure and a catalytic material deposited on said support structure, wherein said support structure comprises a metal oxide felt material and said catalytic material is selected from the group consisting of metals, metal oxides, metal sulfides, mixed metal oxides, mixed metal sulfides.

2. The catalytic process of claim 1 wherein said metal oxide felt material is selected from the group consisting of ZrO₂, CeO₂, Ce₂O₃, TiO₂, Nb₂O₅, Y₂O₃, B₂O₃, HfO₂, Al₂O₃, Al₂O₃—SiO₂, HfO₂—CeO₂, Yb₂O₃—CeO₂, Sm₂O₃—CeO₂, and mixtures and solid solutions thereof.

3. The catalytic process of claim 1 wherein said catalytic material is selected from the group consisting of metals, metal oxides, metal sulfides, mixed metal oxides, mixed metal sulfides.

4. The catalytic process of claim 1 wherein said catalytic material comprises a noble metal and promoters and stabilizers thereof.

5. The catalytic process of claim 1 wherein said catalytic process is a dehydrogenation or hydrogenation reaction.

6. The catalytic process of claim 5 wherein said dehydrogenation converts light paraffins to corresponding light olefins.

7. The catalytic process of claim 5 wherein said hydrogenation reaction converts dienes to a corresponding olefin.

8. The catalytic process of claim 5 wherein said feed for said hydrogenation reaction is a monocarboxylic acid, dicarboxylic acid, multicarboxylic acid or mixtures thereof.

9. The catalytic process of claim 1 wherein said catalytic process is an oxidation of hydrocarbons and said catalyst material comprises one or more metal oxides.

10. The catalytic process of claim 1 wherein said feed comprises one or more light olefins and said catalytic process comprises reacting said one or more light olefins with a catalytic material for in an epoxidation reaction.

11. The catalytic process of claim 10 wherein said catalytic material comprises silver and promoters and stabilizers for said catalytic material.

12. The catalytic process of claim 1 wherein said catalytic material comprises a metal oxide for metal sulfide phase formation in a reducing environment for removal of S-compounds from a gas stream containing said S-compounds and wherein about 10% to about 100% of said metal oxide is converted to a metal sulfide.

13. The catalytic process of claim 1 wherein said catalytic material comprises a metal oxide for metal sulfate phase formation in an oxidizing environment for removal of S-compounds from a gas stream containing S-compounds and wherein about 10 to 100% of said metal oxide is converted to a metal sulfate.

14. The catalytic process of claim 1 wherein said metal oxide felt material comprises layers having a thickness from about 0.25 to about 6.35 mm.

15. The catalytic process of claim 1 wherein said metal oxide felt material has a bulk porosity from about 50 to 100%.

16. The catalytic process of claim 1 wherein said metal oxide felt material has a bulk porosity from about 88 to 96%.

17. The catalytic process of claim 1 wherein said metal oxide felt material has a bulk density of about 128 to 1073 grams/liter.

18. The catalytic process of claim 1 wherein said metal oxide felt material has a melting point between about 1500° and 5000° C.
